(19) United States
(12) Patent Application Publication
(10) Pub. No.: US 2006/0004230 A1
(43) Pub. Date: Jan. 5, 2006

(54) Process for the preparation of terbinafine and salts thereof

(76) Inventors: Joseph Kaspi, Givatayim (IL); Oded Arad, Rehovot (IL); Oded Friedman, Talmey Yechiel (IL); Iosef Manascu, Omer (IL); Tamir Fizitzki, Beer Sheva (IL); Edna Danon, Meitar (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(21) Appl. No.: 10/879,549

(22) Filed: Jun. 30, 2004

(51) Int. Cl. A61K 31/137; C07C 29/10

(52) U.S. Cl. 564/373; 514/649

(57) ABSTRACT

A process for the preparation of Terbinafine and salts thereof by reacting 1-chloro-6,6-dimethylhept-2-en-4-yne and N-methyl-N-(1-naphthylmethyl)amine in a basic aqueous medium is disclosed. Also disclosed is a process for the preparation of 1-chloro-6,6-dimethylhept-2-en-4-yne.

### FIELD AND BACKGROUND OF THE INVENTION

The present invention relates to the field of synthetic chemistry, and more particularly, to a process for the preparation of Terbinafine and Terbinafine salts, especially Terbinafine HCl. The present invention also relates to a process for the preparation of 1-chloro-6,6-dimethyl-2-hepten-4-yne.

Terbinafine (I) (trans-N-(6,6-dimethyl-2-hepten-4-ynyl)-N-methyl-1-naphthylmethyl amine) is an antimycotic agent that inhibits squalene epoxidase thus preventing fungal cells from making ergosterol, a major component of fungal cell walls.

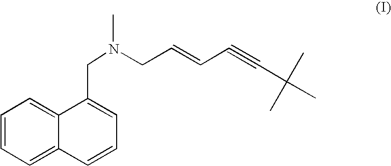

Both oral and topical Terbinafine HCl compositions are prescribed for the treatment of fungal infections from dermatophytes including Tinea corporis, Tinea cruris, Tinea pedis, Tinea mannum and Tinea unguigum. Terbinafine HCl is also prescribed to treat infections by Candida albicans, Epidermophyton floccosum and Scopulariopsis brevicaulis.

In the art, a number of processes for the preparation of Terbinafine (I) and Terbinafine HCl have been taught. It is important to note that one of the crucial issues that must be addressed when preparing Terbinafine is the separation of the Terbinafine from the usually co-prepared respective cis isomer (II):

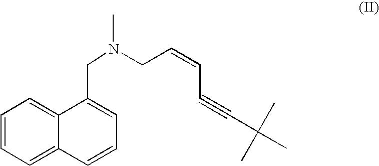

A number of processes for the preparation of Terbinafine (I) involve the coupling of N-methyl-1-naphthylmethyl amine (III): 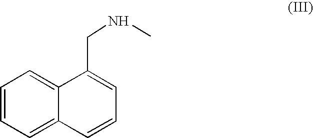
with a 1-X-6,6-dimethyl-2-hepten-4-yne (IV) where X is a leaving group: 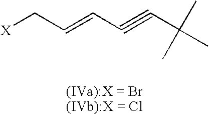
as described in Scheme 1.

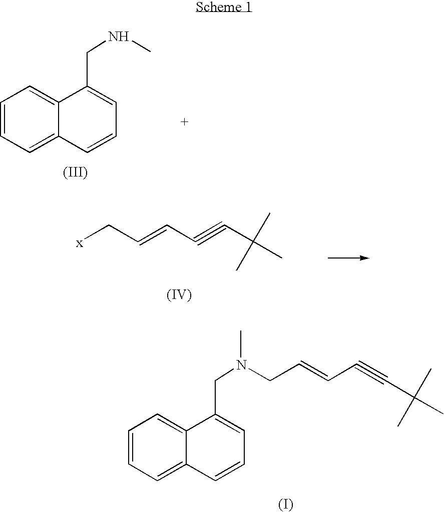

The preparation of 1-Chloro-6,6-dimethyl-2-hepten-4-yne (IVb) by chlorination of 6,6-dimethylhept-1-en-4-yn-3-ol (V) is taught, for example, in EP Patent No. 0 341 048 B1, U.S. Pat. No. 6,570,044, PCT/HU99/00071 published as WO 01/28976 and in Chinese Patent Application No. CN 01139198.7. 6,6-Dimethylhept-1-en-4-yn-3-ol (V) is generally prepared by the condensation of acrolein with t-butylacetylene, as is described, for example, in U.S. Pat. No. 6,570,044.

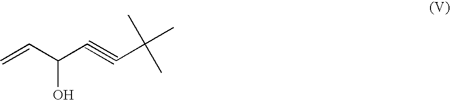

In European patent EP 0 024 587 B1, a 5% molar excess of N-methyl-1-naphthylmethyl amine (III) is reacted with a 3:1 trans/cis isomer mixture of 1-bromo-6,6-dimethyl-2-hepten-4-yne (IVa) in dimethylformamide (DMF), in the presence of K₂CO₃, at room temperature overnight. The DMF is evaporated and the residue partitioned in diethyl ether and saturated aqueous NaHCO₃. Terbinafine (I) is separated from the other reaction products found in the diethyl ether phase, including the Terbinafine cis isomer (II), by chromatography. Terbinafine HCl is prepared by treating pure Terbinafine (I) with a 4N HCl ethanolic solution. EP 0 024 587 B1 is silent with respect to the yield of Terbinafine (I) obtained by this process.

In Stütz et al. (J. Med. Chem. 1984, 27, 1539-1543), the process described in European Pat. No. EP 0 024 587 B1 is repeated, resulting in a 72.4% yield relative to the bromo-6,6-dimethyl-2-hepten-4-yne (IVa). However, instead of chromatographic purification, the diethyl ether is evaporated and the product-containing residue is dissolved in ethanol. Terbinafine (I) and the cis isomer (II) in the ethanolic solution are converted to the respective HCl salts by addition of an ethanolic HCl solution. The solvents are removed by evaporation and Terbinafine HCl is separated from the cis isomer (II) by crystallization from isopropanol/ether, giving a 43.5% yield (relative to IVb) and a recovery of 80% of the Terbinafine (I) in the product-containing residue as Terbinafine HCl.

In Swiss Pat. No. CH 678 527, the hydrochloride salt of N-methyl-1-naphthylmethyl amine (III) is reacted with a 14% excess of a 3:1 trans/cis isomer mixture of 1-bromo-6,6-dimethyl-2-hepten-4-yne (IVa) in a 30% NaOH aqueous solution at a temperature of between about 100° C. and 105° C. for three hours. The raw reaction products, including a 3:1 ratio of Terbinafine (I) to the cis isomer (II), are extracted with toluene as free bases. The yield of Terbinafine (I) is 66.5% relative to N-methyl-1-naphthylmethyl amine (III). The toluene is evaporated and the resulting residue (containing 56.6% Terbinafine (I)) dissolved in anhydrous ethyl acetate. Crystallization of Terbinafine (I) as Terbinafine HCl is initiated by acidification of the ethyl acetate to pH 2 with gaseous HCl. It is stated in CH 678 527 that 86.2% of the Terbinafine in the reaction product was recovered as pure recrystallized Terbinafine HCl.

The processes of preparing Terbinafme (I) taught in European patent EP 0 024 587 B1, Swiss patent CH 678 527 and Stütz et al. J. Med. Chem. 1984, 27, 1539-1543 all couple 1-bromo-6,6-dimethyl-2-hepten-4-yne (IVa) to N-methyl-1-naphthylmethyl amine (III). Due to usage of corrosive and toxic phosphorous tribromide as a reagent in the synthesis of 1-bromo-6,6-dimethyl-2-hepten-4-yne (IVa), its relative instability and the low reaction temperatures required, these processes are disadvantageous, particularly as compared with processes involving the chloro intermediate (IVb).

In patent application PCT/HU/00071, published as WO 01/28976, the preparation of Terbinafine (I) is described via the in situ preparation of 1-chloro-6,6-dimethyl-2-hepten-4-yne (IVb) at −2° C. to 0° C. by the reaction of a fivefold excess of HCl with 6,6-dimethylhept-1-en-4-yn-3-ol (V) under an inert atmosphere for 24 hours. The resulting 1-chloro-6,6-dimethyl-2-hepten-4-yne (IVb) 3.4:1 trans/cis isomer mixture is extracted but not isolated in methyl isobutyl ketone. Thereafter, coupling 1-chloro-6,6-dimethyl-2-hepten-4-yne (IVb) with an equimolar amount of N-methyl-1-naphthylmethyl amine (III) is performed in methyl isobutyl ketone, in the presence of water, N,N-diisopropyl ethylamine and tetrabutylammonium iodide, under a nitrogen atmosphere, for 1 hour at between 70° C. and 80° C. Purification of the resulting Terbinafine (I) and separation from the cis isomer (II) is performed by acidification of the product-containing organic phase with aqueous HCl and a plurality of washing steps. Thus, Terbinafine (I) is separated from the cis isomer (II) and simultaneously converted to Terbinafine HCl by the addition of an aqueous HCl solution in an amount sufficient to reduce the pH of the solution to about 1.5-2. When methyl isobutyl ketone was used as the organic solvent, a best yield of 53.4% of Terbinafine HCl relative to the 6,6-dimethylhept-1-en-4-yn-3-ol (V) and of 52.5% relative to the N-methyl-1-naphthylmethyl amine (III) was obtained (see, Example 1 in WO 01/28976). The process taught in WO 01/28976, albeit using the milder chloro intermediate IVb, is therefore inefficient, both in terms of the overall yield and the solvents and reaction conditions used (e.g., inert atmosphere). The use of the organic solvent methyl isobutyl ketone renders this process highly cost-ineffective, as well as environmentally unfriendly. Further, the process requires the use of corrosive N,N-diisopropyl ethylamine as a base and tetrabutylammonium iodide as a catalyst, both are known as hazardous reagents.

In Chinese Patent Application No. CN 01139198.7, Terbinafine (I) is prepared by reacting N-methyl-1-naphthylmethyl amine (III) with trans-1-chloro-6,6-dimethyl-2-hepten-4-yne (IVb) in dimethyl formamide (DMF) with sodium carbonate at between 80° C. and 100° C. A maximal yield of 84.4% relative to the 1-chloro-6,6-dimethyl-2-hepten-4-yne (IVb) is reported (see Example 2). 1-chloro-6,6-dimethyl-2-hepten-4-yne (IVb) is prepared through 6,6-dimethylhept-1-en-4-yn-3-ol (V) as a non-isolated intermediate from acrolein and t-butyl acetylene. Subsequently, the trans 1-chloro-6,6-dimethyl-2-hepten-4-yne (IVb) is isolated from the reaction mixture through a not-defined rectification step. The best reported yield of trans 1-chloro-6,6-dimethyl-2-hepten-4-yne (IVb) relative to acrolein is 58.2%.

The process requires the use of expensive, unpleasant and environmentally unfriendly reagents and solvents such as DMF. Additionally, DMF has a high boiling point and is therefore less suitable for industrial applications due to the high-energy requirement for evaporation and recycling. Furthermore, the steps required for purifying the 1-chloro-6,6-dimethyl-2-hepten-4-yne (IVb) are expensive, time-consuming, and difficult to implement on an industrial scale. The direct and indirect costs of the process are so high that the advantages achieved from the isomer ratio are overcome.

A number of different approaches for the preparation of Terbinafine taught in the art do not involve the coupling of N-methyl-1-naphthylmethyl amine (III) with a 1-X-6,6-dimethyl-2-hepten-4-yne (IV).

In a second process taught in European patent EP 0 024 587 B1, N-methyl-N-(1-naphthylmethyl)-6,6-dimethylhept-2-4-diynyl-1-amine is reduced by catalytic hydrogenation to give Terbinafine (I). N-methyl-N-(1-naphthylmethyl)-6,6-dimethylhept-2-4-diynyl-1-amine is prepared by coupling N-methyl-N-naphthylmethyl amine (III) with 6,6-dimethylhept-2-4-diyne in the presence of copper chloride, or from the acetylene coupling reaction between N-methyl-N-naphthylmethylpropargyl amine and tert-butylacetylene bromide. A similar process is taught in Granitzer et al. Tetra. Lett. 1979, 34, 3145.

In a third process taught in European patent EP 0 024 587 B1, Terbinafine (I) is prepared by reductive amination of N-methyl-N-naphthylamine (III) with trans-6,6-dimethylhept-2-en-4-yn-1-al in the presence of formaldehyde and sodium borohydride. A similar process is taught in Granitzer et al. Tetra. Lett. 1979, 34, 3145

In a first process for the preparation of Terbinafine (I) taught in U.S. Pat. No. 5,817,875, N-methyl-1-naphthylmethyl amine (III) is converted to an epoxide by reaction with excess epichlorohydrin. The epoxide is reacted with lithium. t-butylacetylide in the presence of a Lewis acid to give 6,6-dimethylhept-1-en-4-yn-3-ol to yield a mixture of isomers of the secondary alcohol N-(6,6-dimethyl-2-ol-4-ynyl)-N-methyl-1-naphthylmethylamine which is subsequently dehydrated to yield a mixture of Terbinafine (I) and the cis isomer (II).

In a second process for the preparation of Terbinafine (I) taught in U.S. Pat. No. 5,817,875, N-methyl-1-naphthylmethyl amine (III) is converted, by reaction with a bromoacetaldehyde dialkylacetal in the presence of a base to obtain a tertiary amine, which undergoes acid hydrolysis to obtain an N-methyl N-1-naphtylmethyl N-methylaldehyde which is subsequently reacted with a 3,3-dimethyl butyne phosphorous compound through a Wittig reaction in the presence of a base to yield a mixture of Terbinafine (I) and the cis isomer (II).

In Spanish patent ES 550,015 1-chloro-6,6-dimethylhept-2-en-4-yne (IVa) is reacted with large excess of methylamine to yield N-methyl-N-(E)-(6,6-dimethylhept-2-en-4-ynyl)amine. The N-methyl-N-(E)-(6,6-dimethylhept-2-en-4-ynyl)amine is reacted with 1-chloromethylnaphthalene in a Na₂CO₃ water/ethanol solution to form Terbinafine (I). Isolated Terbinafine (I) was dissolved in isopropanol through which HCl was bubbled to yield Terbinafine HCl. This process lacks an enabling description because the preparation of N-methyl-N-(E)-(6,6-dimethylhept-2-en-4-ynyl)amine, essential to this process, is not detailed and usage of large excess methylamine is very expensive.

In a first process described in European Patent 0 421 302 B1, trans-N-(3-chloro-2-propenyl)-N-methyl-1-naphthylmethyl amine and t-butylacetylene were coupled in a tetrahydrofuran solution in the presence of copper iodide, butylamine and a palladium catalyst yielding Terbinafine (I). Terbinafine (I) was isolated using chromatography, dissolved in ethanol to which a 23% HCl methanol solution was added. Distillation of the solvent yielded Terbinafine HCl crystals. The trans-N-(3-chloro-2-propenyl)-N-methyl-1-naphthylmethyl amine was prepared by coupling N-methyl-1-naphthylmethyl amine (III) and 1,3-dichloropropene (E/Z=9/1) in dimethyl sulfoxide in the presence of potassium carbonate. A similar process is described in Gotteland et al. Tetra. Lett. 1996, 37(1), 57.

In a second process described in European Patent 0 421 302 B1, 1-chloromethylnaphthalene is reacted with trans-N-(6,6-dimethyl-2-hepten-4-ynyl)methylamine HCl in a dimethylsulfoxide solution in the presence of potassium carbonate. Upon reaction completion, ethyl acetate was added followed by an aqueous HCl solution. Evaporation of the solvents led to crystallization of Terbinafine HCl.

Thus, all of the presently known processes for the preparation of Terbinafine involve environmentally unfriendly solvents and other reagents, usage of excessive amounts of hazardous reagents such as 3,3-dimethylbutyne and epichlorohydrin (the latter is a toxic and carcinogenic material) and/or laborious isolation of the final product by column chromatography.

There is thus a widely recognized need for, and it would be highly advantageous to have, an improved process for the preparation of Terbinafine devoid of at least some of the disadvantages of processes known in the art.

### SUMMARY OF THE INVENTION

The present invention successfully addresses the above-recited need by providing an innovative process for the preparation of Terbinafine (I) and salts thereof. Further, the present invention successfully addresses the above-recited need by providing an innovative process for the preparation of 1-chloro-6,6-dimethyl-2-hepten-4-yne (IVb).

According to the teachings of the present invention there is provided a process of preparing Terbinafine (I) and/or a salt thereof, the process comprising: providing 1-chloro-6,6-dimethyl-2-hepten-4-yne (IVb) and reacting the 1-chloro-6,6-dimethyl-2-hepten-4-yne (IVb) with N-methyl-1-naphtylmethylamine (III), in an aqueous reaction medium, thereby obtaining the Terbinafine (I). In embodiments of the present invention, the aqueous reaction medium comprises at least 25%, at least 50%, or even at least 75% by weight water.

According to a feature of the present invention, subsequent to the reacting, the obtained Terbinafine (I) is contacted with HCl, to obtain Terbinafine HCl salt as a precipitate.

According to a feature of the present invention, the obtained Terbinafine HCl is re-crystallized to obtain Terbinafine HCl salt having a pharmaceutical quality.

According to a feature of the present invention, the pharmaceutically pure Terbinafine HCl salt is converted to Terbinafine (I), thereby obtaining Terbinafine (I) having a pharmaceutical quality.

Generally, the aqueous reaction medium comprises a base. Suitable bases include but are not limited to sodium carbonate, potassium carbonate, sodium bicarbonate and potassium bicarbonate. Sodium carbonate is a preferred base. In embodiments of the present invention, the concentration of the base preferably ranges between about 50 and about 400, more preferably between about 100 and about 300 and more preferably between about 125 and about 200 grams per liter of the aqueous reaction medium.

According to a feature of the present invention, reacting the 1-chloro-6,6-dimethyl-2-hepten-4-yne (IVb) with N-methyl-1-naphtylmethylamine (III) comprises: providing an aqueous solution containing the N-methyl-1-naphtylmethylamine (III); and reacting the 1-chloro-6,6-dimethyl-2-hepten-4-yne (IVb) with the aqueous solution containing the N-methyl-1-naphtylmethylamine.

Suitable concentrations of N-methyl-1-naphthylmethyl amine (III) in the aqueous solution ranges between about 0.1 M and about 20 M, between about 0.5 M and about 15 M and between about 2 M and about 10 M. Generally the molar ratio between the 1-chloro-6,6-dimethyl-2-hepten-4-yne (IVb) and the N-methyl-1-naphtylmethylamine (III) ranges between about 1:0.9 and 1:1.1 or between about 1:0.95 and 1:1.05.

In embodiments of the present invention the reacting is effected by adding the 1-chloro-6,6-dimethyl-2-hepten-4-yne (IVb) to the aqueous solution. It is preferred to heat the aqueous solution prior to the reacting to a temperature of at least 40° C., of at least 50° C., of at least 65° C., of at least 70° C. or even of at least 75° C.

In a feature of the present invention, subsequent to the reacting, an organic solution containing the Terbinafine (I) is provided. In an embodiment of the present invention, providing the organic solution containing the Terbinafine (I) comprises: separating a first amount of the Terbinafine (I) from the aqueous reaction medium. In an embodiment of the present invention, subsequent to the separating, the aqueous reaction medium is contacted with an organic extraction solution, to thereby extract the Terbinafine (I) into the organic extraction solution. Generally the organic extraction solution comprises at least one organic solvent selected from the group consisting of an ether, a linear alkane, a cycloalkane, a branched alkane, an aromatic solvent, an ester, a ketone, a halogenated hydrocarbon, a nitrile and any mixture thereof as detailed below. In a preferred embodiment of the present invention the at least one organic solvent comprises toluene and preferably comprises more than 50% toluene, more than 80% toluene and even more than 95% toluene.

According to an embodiment of the present invention, the separated Terbinafine and the organic extraction solution are combined, to thereby obtain the organic solution containing the Terbinafine.

As stated hereinabove, according to a feature of the present invention, the obtained organic solution containing the Terbinafine (I) is contacted with HCl, to obtain Terbinafine HCl salt as a precipitate. In an embodiment of the present invention the contacting is effected by contacting the organic solution containing the Terbinafine (I) with an aqueous HCl solution. In some embodiments the aqueous HCl solution further comprises ethanol. In another embodiment of the present invention, the contacting is effected by contacting the organic solution containing the Terbinafine (I) with gaseous HCl.

As stated hereinabove, according to a feature of the present invention, the Terbinafine HCl precipitate is re-crystallized to obtain Terbinafine HCl salt having a pharmaceutical quality. According to a feature of the present invention, the re-crystallization is performed in an organic solvent. Generally, the Terbinafme HCl is completely dissolved in the organic solvent at a first temperature and then the organic solvent is allowed to cool to a second temperature, leading to crystallization of the Terbinafine HCl while the greatest share of the Terbinafine cis isomer (II) remains in solution. Clearly, the first temperature (generally reflux temperature) is higher than the second temperature. Preferred organic solvents for re-crystallization of Terbinafine HCl include organic solvents selected from the group consisting of methanol, ethanol, n-propanol, isopropanol, n-butanol, sec-butanol, ethyl acetate, isopropyl acetate, n-butyl acetate, isobutyl acetate, acetonitrile and any mixture thereof. In a preferred embodiment of the present invention the at least one organic solvent comprises isopropanol and preferably comprises more than 50% isopropanol, more than 80% isopropanol and even more than 95% isopropanol.

The 1-chloro-6,6-dimethyl-2-hepten-4-yne (IVb) provided for use in the process for preparing Terbinafine (I) of the present invention is available from a number of sources and can be prepared using a number of processes. The preferred process for providing 1-chloro-6,6-dimethyl-2-hepten-4-yne (IVb) is the process of the present invention, detailed hereinbelow.

According to the teachings of the present invention, there is provided a process of preparing 1-chloro-6,6-dimethyl-2-hepten-4-yne (IVb), the process comprising providing 6,6-dimethylhept-1-en-4-yn-3-ol (V) and reacting the 6,6-dimethylhept-1-en-4-yn-3-ol (V) with a chlorinating agent, in an aqueous reaction medium, to thereby obtain the 1-chloro-6,6-dimethyl-2-hepten-4-yne (IVb). Generally, reacting is performed at a temperature greater than about 0° C., greater than about 2° C., greater than about 5° C. and even greater than about 10° C.

In an embodiment of the present invention, providing the 6,6-dimethylhept-1-en-4-yn-3-ol (V) comprises providing t-butylacetylide and reacting the t-butylacetylide with acrolein. In an embodiment of the present invention, the reacting is performed at a temperature of between about 0° C. and about 5° C. In an embodiment of the present invention, providing the t-butylacetylide comprises reacting t-butylacetylene and an organomagnesium compound of the Grignard type, such as ethylmagnesium bromide.

In an embodiment of the present invention, the 6,6-dimethylhept-1-en-4-yn-3-ol (V) is provided in a solution with a water-miscible organic solvent. Suitable water-miscible organic solvents include but are not limited to methanol, ethanol, n-propanol, isopropanol, n-butanol, sec-butanol and any mixture thereof. The concentration of the 6,6-dimethylhept-1-en-4-yn-3-ol (V) in such a substrate solution is typically between about 0.1 and about 20 M, preferably between about 0.5 and about 15 M, and more preferably between about 2 and about 10 M.

In an embodiment of the present invention, prior to the reacting, a solution containing the chlorinating agent is provided. In an embodiment of the present invention, the solution comprises at least 25%, at least 50% or even at least 75% by weight water. In an embodiment of the present invention, the aqueous reaction medium comprises at least one water-miscible organic solvent. Suitable water-miscible organic solvents include but are not limited to methanol, ethanol, n-propanol, isopropanol, n-butanol, sec-butanol and any mixture thereof. A preferred water-miscible organic solvent is ethanol.

According to a feature of the present invention, the chlorinating agent comprises a mixture of PCl₃ and HCl. In an embodiment of the present invention, prior to reacting, a solution including HCl and PCl₃ is provided. In an embodiment of the present invention the solution comprises water. In an embodiment of the present invention the solution further comprises ethanol. In an embodiment of the present invention, the concentration of the PCl₃ in the solution is between about 0.1 M and about 3 M, preferably between about 0.2 M and about 2 M and more preferably between about 0.5 M and about 1 M. In an embodiment of the present invention, the concentration of HCl in the solution is greater than about 20%, preferably greater than about 24%, more preferably greater than about 28%, more preferably greater than about 30%, more preferably greater than about 32%, more preferably greater than about 34%, more preferably greater than about 36%, with a concentration of between about 36.5% and 37.5% HCl being the presently most preferred.

According to a feature of the present invention, subsequent to the reacting, an organic extraction solution is contacted with the aqueous reaction medium containing the 1-chloro-6,6-dimethyl-2-hepten-4-yne (IVb) to thereby provide an organic solution containing the 1-chloro-6,6-dimethyl-2-hepten-4-yne. Generally the organic extraction solution comprises at least one organic solvent selected from the group consisting of an ether, a linear alkane, a cycloalkane, a branched alkane, an aromatic solvent, an ester, a ketone, a halogenated hydrocarbon, a nitrile and any mixture thereof as detailed hereinbelow. In a preferred embodiment of the present invention the at least one organic solvent comprises hexane and preferably comprises more than 50% hexane, more preferably more than 80% hexane and even more preferably more than 95% hexane.

Extraction solutions, formulation thereof and uses thereof are known to one skilled in the art. In general, an extraction solution is a solvent or mixture of solvents that upon mixing with a reaction solution lead to the formation of at least two phases, one phase wherein the desired product or products are substantially dissolved and at least one phase wherein other products and/or reagents are dissolved. It is generally preferred to formulate an extraction solution wherein only two phases are formed. It is further preferable that as great a proportion as possible of the desired product or products be found in on of the two phases.

In both processes of the present invention, the preparation of 1-chloro-6,6-dimethyl-2-hepten-4-yne (IVb) and the preparation of Terbinafine (I) and salts thereof, the reaction takes place in an aqueous reaction medium and the desired reaction product is extracted from the aqueous solution using an organic extraction solution. Suitable organic extraction solutions are solutions that, when mixed with the respective aqueous reaction solutions, form a product-containing organic phase in addition to an aqueous phase substantially formed by the aqueous reaction phase. Preferred organic extraction solutions comprise solvents including but are not limited to solvents such as ethers (e.g. diethyl ether, diisopropyl ether, methyl-t-butyl ether, THF), linear or branched alkanes and cycloalkanes (e.g., n-pentane, n-hexane, n-heptane, n-octane, isooctane, n-nonane, n-decane, cyclohexane), aromatic solvents (e.g. benzene, toluene, xylenes), esters and ketones (e.g. ethyl acetate, isopropyl acetate, n-butyl acetate, isobutyl acetate, acetone, methyl isobutyl ketone, acetonitrile), halogenated hydrocarbons (e.g. CHCl₃, CH₂Cl₂, CH₃Cl) and any mixture thereof.

Unless otherwise defined, all technical and scientific terms used herein have the same meaning as commonly understood by one of ordinary skill in the art to which this invention belongs. Although processes and materials similar or equivalent to those described herein can be used in the practice or testing of the present invention, suitable processes and materials are described below. All publications, patent applications, patents, and other references mentioned herein are incorporated by reference in their entirety. In case of conflict, the patent specification, including definitions, will control. In addition, the materials, processes, and examples are illustrative only and not intended to be limiting.

### DESCRIPTION OF THE PREFERRED EMBODIMENTS

The present invention is of a process for the preparation of Terbinafine (I) and Terbinafine salts, especially Terbinafine HCl. The present invention also provides a process for the preparation of a Terbinafine precursor, 1-chloro-6,6-dimethylhept-2-en-4-yne (IVb).

The principles, uses and implementations of the present invention are better understood with reference to the accompanying descriptions and examples.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

As used herein, the term “comprising” means that other steps and ingredients which do not affect the final result can be added. This term encompasses the terms “consisting of” and “consisting essentially of”.

The phrase “consisting essentially of” means that the composition may include additional ingredients, but only if the additional ingredients do not materially alter the basic and novel characteristics of the claimed compositions or methods.

The term “method”, which is also referred to herein interchangeably as “process”, refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein throughout, the term “weight percentage(s)” describes the weight percentage(s) of an ingredient of the total weight of a composition containing the ingredient.

As used herein throughout the term “about” refers to ±10%.

As used herein throughout, the term “pharmaceutical quality” has the same meaning as found in the Pharmacopeia of the United States, that is that a substance (e.g., Terbinafine or a Terbinafine salt) has purity characteristics that conform to drug regulations assuring that the substance meets the requirements of the act as to safety and meets the quality it is represented to possess. Typically, a substance (e.g., drug) having a pharmaceutical quality is a substance having less than 0.5%, preferably less than 0.45%, more preferably less than 0.40%, more preferably less than 0.35%, more preferably less than 0.30%, more preferably less than 0.25%, more preferably less than 0.20%, and most preferably less than 0.15% of an isomer thereof. In addition, typically, a substance (e.g., drug) having a pharmaceutical quality is a substance having less than 1.0%, preferably less than 0.95%, more preferably less than 0.90%, more preferably less than 0.85%, more preferably less than 0.80%, more preferably less than 0.75%, more preferably less than 0.70%, more preferably less than 0.65%, more preferably less than 0.60%, more preferably less than 0.55%, more preferably less than 0.50%, more preferably less than 0.45%, more preferably less than 0.40%, more preferably less than 0.35%, more preferably less than 0.30%, more preferably less than 0.25%, more preferably less than 0.20% and more preferably less than 0.15% total content of impurities.

In a preferred embodiment of the present invention, a product having a pharmaceutical quality is a product containing no more than 0.15% of the respective Z-isomer and having a total content of impurities of no more than 0.5%.

According to the present invention Terbinafine (I) is prepared by reacting 1-chloro-6,6-dimethyl-2-hepten-4-yne (IVb) with N-methyl-1-naphthylmethyl amine (III) as depicted in scheme 2.

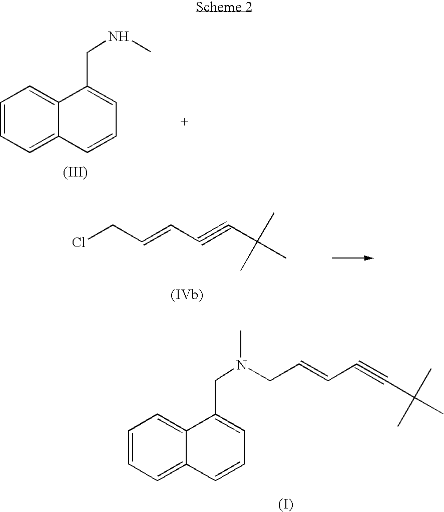

As shown herein, Terbinafine (I) or its HCl salt are obtained conforming to pharmaceutical quality by reacting an isomer mixture of 1-chloro-6,6-dimethyl-2-hepten-4-yne (IVb) with N-methyl-1-naphthylmethyl amine (III), converting the resulting isomer mixture of Terbinafine (I) to its HCl salt and separating the relatively pure Terbinafine HCl from the cis isomer (II) by recrystallization. The relative ease of separating Terbinafine HCl from the cis isomer (II) is advantageous over the ineffective process of producing pure trans 1-chloro-6,6-dimethyl-2-hepten-4-yne (IVb) as a precursor.

Reacting 1-chloro-6,6-dimethyl-2-hepten-4-yne With N-methyl-1-naphthylmethyl amine:

The process of the present invention for preparing Terbinafine (I) and/or salts thereof is based on reacting 1-chloro-6,6-dimethyl-2-hepten-4-yne (IVb) with N-methyl-1-naphthylmethyl amine (III) as depicted in scheme 2 hereinabove. According to the present invention, Terbinafine (I) is produced almost quantitatively by the reaction of 1-chloro-6,6-dimethyl-2-hepten-4-yne (IVb) with N-methyl-1-naphthylmethyl amine (III) in a basic aqueous reaction medium. The N-methyl-1-naphthylmethyl amine (III) is provided as a free base or as any convenient addition salt, such as N-methyl-1-naphthylmethyl amine HCl.

The aqueous reaction medium preferably comprises at least 25% by weight water, at least 50% by weight water, and even at least 75% by weight water.

The aqueous reaction medium of the present invention is preferably basic. Generally the aqueous reaction medium is made basic by the addition of a base before, during or after, preferably before, the 1-chloro-6,6-dimethyl-2-hepten-4-yne (IVb) and the N-methyl-1-naphthylmethyl amine (III) are reacted. The amount of base added is between about 50 and 400 gram per liter, more preferably between about 100 and 300 gram per liter, and even more preferably between about 125 and 200 gram per liter of the aqueous reaction medium. Preferred added bases include, but are not limited to, sodium carbonate, potassium carbonate sodium bicarbonate and potassium bicarbonate. In a preferred embodiment of the present invention, sodium carbonate is the base added.

In an embodiment of the present invention, the molar amount of the 1-chloro-6,6-dimethyl-2-hepten-4-yne (IVb) used in the reaction differs within about 10% percent (ratio of [III]:[IVb] between 1:0.9 and 1:1.1), preferably within about 5% (ratio [III:[IVb] between 1:0.95 and 1:1.05, from the molar amount of the N-methyl-1-naphthylmethyl amine (III) used.

In one embodiment of the present invention, the N-methyl-1-naphthylmethyl amine (III) is dissolved in an aqueous solution prior to the reaction with the 1-chloro-6,6-dimethyl-2-hepten-4-yne. In such a case, the concentration of the N-methyl-1-naphthylmethyl amine (III) in the aqueous solution is preferably between about 0.1 and 20 M, more preferably between about 0.5 and 15 M, and even more preferably between about 2 and 10 M.

In one embodiment of the present invention, the N-methyl-1-naphthylmethyl amine (III) is provided in an aqueous solution, which serves as the aqueous reaction medium, such that the reaction with the 1-chloro-6,6-dimethyl-2-hepten-4-yne (IVb) is effected by adding the 1-chloro-6,6-dimethyl-2-hepten-4-yne (IVb) to the aqueous solution containing the N-methyl-1-naphthylmethyl amine (III).

The reaction generally occurs at elevated temperatures. Preferably the reaction is performed at a temperature greater than about 40° C., more preferably greater than about 50° C., more preferably greater than about 65° C., more preferably greater than about 70° C. or even more preferably greater than about 75° C., for at least part of the time the reaction takes place.

In one embodiment of the present invention, the aqueous solution containing the N-methyl-1-naphthylmethyl amine (III), is heated to the reaction temperature prior to the addition of 1-chloro-6,6-dimethyl-2-hepten-4-yne (IVb).

The currently known best mode of preparing Terbinafine (I) according to the teachings of the present invention comprises:
a. providing an aqueous reaction medium by combining a base, preferably sodium carbonate, and N-methyl-1-naphthylmethyl amine (III) or a salt thereof in water;
b. adding an amount of 1-chloro-6,6-dimethyl-2-hepten-4-yne (IVb) to the aqueous reaction medium; and
c. reacting the N-methyl-1-naphthylmethyl amine (III) with the 1-chloro-6,6-dimethyl-2-hepten-4-yne (IVb) so as to obtain the Terbinafine (I).

Preferably, the concentration of the N-methyl-1-naphthylmethyl amine (III) in the aqueous reaction medium is between about 2M and 10M.

The amount of base added is preferably between about 125 and 200 gram per liter of the aqueous reaction medium.

Preferably the amount of 1-chloro-6,6-dimethyl-2-hepten-4-yne (IVb) added differs within about 10% percent of the amount of the N-methyl-1-naphthylmethyl amine (III) in the aqueous reaction medium.

Preferably during the reaction, the temperature of the aqueous reaction medium is maintained to be greater than 70° C.

Purification of Reaction Products:

Subsequent to reacting 1-chloro-6,6-dimethyl-2-hepten-4-yne (IVb) with N-methyl-1-naphthylmethyl amine (III), it is preferred to purify the resulting Terbinafine (I). If an isomeric mixture of 1-chloro-6,6-dimethyl-2-hepten-4-yne (IVb) is used in the reaction, it is preferred to separate the Terbinafine (I) from the co-produced cis isomer (II). Such a separation has been described the art.

For example, according to the teachings of WO 01/28976, the reaction products are extracted into methyl isobutyl ketone (or other organic solvents listed therein) and the Terbinafine (I) separated from the cis isomer (II) by crystallization as the HCl salt by the addition of aqueous HCl to a pH of 1.5-2.0.

According to the teachings of Swiss Patent CH 678 527, the reaction products are extracted into toluene, the toluene removed by evaporation, and Terbinafine HCl recovered from the product-containing residue by dissolution in ethyl acetate and crystallization using gaseous HCl added to a pH of about 2.

According to the teachings of Stütz et al. J. Med. Chem. 1984, 27, 1539-1543, the reaction products are extracted into an organic solvent, the organic solvent removed by evaporation, and Terbinafine (I) and cis isomer (II) converted to the corresponding hydrochloride salts in ethanol, which is thereafter removed by evaporation. The Terbinafine HCl is isolated from the hydrochloride salt mixture by recrystallization from isopropanol/ether.

Contrary to the processes described above, according to the process of the present invention, the purification of Terbinafine is effected by providing a Terbinafine HCl salt as a precipitate and re-crystallizing the Terbinafine HCl to thereby isolate the cis isomer and provide a purified Terbinafine HCl salt, as follows:

When the reaction described above is completed, an organic solution containing the Terbinafine (I) is preferably provided. Since Terbinafine (I) is not soluble in the aqueous reaction medium, providing an organic solution containing the Terbinafine (I) optionally includes separating a first amount of the Terbinafine (I) from the aqueous reaction medium, using techniques known in the art. Providing an organic solution containing the Terbinafine (I) optionally further includes contacting the aqueous reaction medium with an organic extraction solution so as to extract the Terbinafine (I) into the organic extraction solution. Suitable organic extraction solutions are detailed above, but in a preferred embodiment of the present invention the organic extraction solution comprises toluene, and preferably comprises more than 50% toluene, more than 80% toluene and even more than 95% toluene.

Once the organic extraction solution containing the Terbinafine (I) is obtained, it is preferred to combine the first amount of the Terbinafine (I) and the organic extraction solution so as to obtain the desired organic solution containing the Terbinafine (I).

The organic solution containing Terbinafine (I) also contains other soluble compounds including the cis isomer (II). Separation of the Terbinafine (I) from other reaction products including at least some of the cis isomer (II) is performed by precipitation of Terbinafine (I) as Terbinafine HCl by contacting the organic solution containing Terbinafine (I) with HCl. Depending on the embodiment, the organic solution containing Terbinafine (I) is contacted with gaseous HCl, with aqueous HCl, ethanolic HCl or a combination thereof.

The precipitated Terbinafine HCl is separated from the organic solution containing Terbinafine (I), for example, by filtering and/or centrifugation.

An advantage of precipitating Terbinafine (I) from the organic solution containing Terbinafine (I) as Terbinafine HCl is that a relatively high proportion of the cis isomer (II) does not precipitate out of the organic solution containing Terbinafine (I). However, generally the precipitated Terbinafine HCl is still not sufficiently pure for pharmaceutical use. Thus, the precipitated Terbinafine HCl is generally purified further. Processes for purification of Terbinafine HCl are known in the art.

According to an embodiment of the present invention, purifying Terbinafine HCl is effected by recrystallization from an organic solvent, such as methanol, ethanol, n-propanol, isopropanol, n-butanol, sec-butanol, ethyl acetate, isopropyl acetate, n-butyl acetate, isobutyl acetate, acetonitrile and any mixture thereof. In a preferred embodiment, the organic solvent comprises isopropanol and preferably comprises more than 50% isopropanol, more than 80% isopropanol and even more than 95% isopropanol.

In a preferred process for recrystallization of Terbinafine HCl, the Terbinafine HCl is entirely dissolved in isopropanol (at a reflux temperature) and crystallizes out when the temperature of the isopropanol is reduced. Thus the Terbinafine HCl is dissolved in an organic solvent, preferably isopropanol, at an elevated temperature. After the Terbinafine HCl is completely dissolved, cooling of the organic solvent leads to precipitation of Terbinafine HCl substantially devoid of the cis isomer (II) or salts thereof, providing Terbinafine (I) pharmaceutical quality, as defined hereinabove.

Once Terbinafine HCl of pharmaceutical quality is isolated, free Terbinafine (I) can be liberated, and if so desired, converted according to processes known in the art to any other desired acid addition salt.

Preparation of 1-chloro-6,6-dimethyl-2-hepten-4-yne:

The present invention provides a process of preparing 1-chloro-6,6-dimethyl-2-hepten-4-yne (IVb) by providing 6,6-dimethylhept-1-en-4-yn-3-ol (V) and reacting the 6,6-dimethylhept-1-en-4-yn-3-ol (V) with a chlorinating agent (preferably a mixture of PCl₃ and HCl) in an aqueous reaction medium so as to obtain 1-chloro-6,6-dimethyl-2-hepten-4-yne (IVb), as depicted in scheme 3.

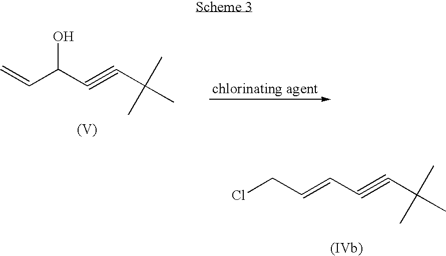

In an embodiment of the present invention, the 6,6-dimethylhept-1-en-4-yn-3-ol (V) is provided by providing t-butylacetylide and reacting the t-butylacetylide with acrolein, preferably at temperature of between about 0° C. and about 5° C. In an embodiment of the present invention, the t-butylacetylide is provided by reacting t-butylacetylene and an organomagnesium compound of the Grignard type, for example, ethylmagnesium bromide.

According to a feature of the present invention, prior to reacting the 6,6-dimethylhept-1-en-4-yn-3-ol (V) with a chlorinating agent, a solution containing the 6,6-dimethylhept-1-en-4-yn-3-ol and a water miscible organic solvent is provided. Suitable water miscible organic solvents include, but are not limited to, methanol, ethanol, n-propanol, isopropanol, n-butanol, sec-butanol and any mixture thereof. In an embodiment of the present invention, the concentration of the 6,6-dimethylhept-1-en-4-yn-3-ol in the solution ranges between about 0.1 and about 20 M, preferably between about 0.5 M and about 15 M and more preferably between about 2 M and 10 M.

In an embodiment of the present invention, prior to reacting the 6,6-dimethylhept-1-en-4-yn-3-ol (V) with a chlorinating agent, a solution comprising the chlorinating agent is provided. According to a feature of the present invention, a preferred chlorinating agent comprises a mixture of PCl₃ and HCl. In an embodiment of the present invention prior to reacting the 6,6-dimethylhept-1-en-4-yn-3-ol (V) with the chlorinating agent, a solution comprising HCl and PCl₃ is provided. Preferably the solution comprises water. In an embodiment of the present invention, the solution further comprises ethanol. In an embodiment of the present invention, the concentration of PCl₃ in the solution is between about 0.1 M and about 3 M, preferably between about 0.2 M and about 2 M, and more preferably between about 0.5 M and about 1 M. In an embodiment of the present invention, the concentration of HCl in the solution is greater than about 20%, preferably greater than about 24%, more preferably greater than about 28%, more preferably greater than about 30%, more preferably greater than about 32%, more preferably greater than about 34% and even more preferably greater than about 36% HCl.

According to a feature of the present invention, the reaction of 6,6-dimethylhept-1-en-4-yn-3-ol (V) with a chlorinating agent occurs in an aqueous reaction medium. According to a feature of the present invention, water makes up at least 25% by weight, at least 50% by weight or even at least 75% by weight of the aqueous reaction medium. In some embodiments the aqueous reaction medium comprises at least one water-miscible organic solvent. Suitable water-miscible organic solvents include but are not limited to methanol, ethanol, n-propanol, isopropanol, n-butanol and isobutanol. A most preferred water-miscible organic solvent is ethanol.

According to a feature of the present invention, the reacting of the 6,6-dimethylhept-1-en-4-yn-3-ol (V) with the chlorinating agent occurs efficiently and in a controlled manner at room temperature. Accordingly, during the reaction, the temperature of the aqueous reaction medium is greater than about 0° C., preferably greater than about 2° C., more preferably greater than about 5° C. and even more preferably greater than about 10° C.

According to a feature of the present invention, once a sufficient amount of 1-chloro-6,6-dimethyl-2-hepten-4-yne (IVb) is prepared, the aqueous reaction medium is contacted with an organic extraction solution, to thereby provide an organic solution containing the 1 -chloro-6,6-dimethyl-2-hepten-4-yne. Suitable organic extraction solutions are detailed above, but in a preferred embodiment of the present invention the organic extraction solution comprises hexane, and preferably comprises more than 50% hexane, more than 80% and even more than 95% hexane.

The currently known best process for preparing 1-chloro-6,6-dimethyl-2-hepten-4-yne (IVb), according to the present invention, therefore involves reacting 6,6-dimethylhept-1-en-4-yn-3-ol (V) with an aqueous PCl₃/HCl solution having at least 25% by weight water. The reaction of the 6,6-dimethylhept-1-en-4-yn-3-ol (V) is nearly quantitative and provides a 3.5:1 trans/cis isomer mixture of 1-chloro-6,6-dimethyl-2-hepten-4-yne (IVb). The reaction is so clean that post-reaction isolation of the 1-chloro-6,6-dimethyl-2-hepten-4-yne (IVb) involves only partitioning the reaction products between an aqueous and a product-containing organic phase followed by evaporation of the organic phase solvent.

The currently known best mode of preparing 1-chloro-6,6-dimethyl-2-hepten-4-yne (IVb) according to the teachings of the present invention comprises:
a. providing an aqueous solution including HCl and PCl₃;
b. providing a 6,6-dimethylhept-1-en-4-yn-3-ol (V) solution; and
c. reacting 6,6-dimethylhept-1-en-4-yn-3-ol (V) solution with the HCl/PCl₃ solution to produce the 1-chloro-6,6-dimethyl-2-hepten-4-yne (IVb).

The most straightforward and industrially applicable process of providing the PCl₃/HCl solution is by dissolving PCl₃ in an aqueous HCl solution. Although any aqueous HCl solution can be used, it is preferred that the HCl solution be a concentrated HCl solution including at least 24% HCl, preferably at least 28% HCl, more preferably at least 30% HCl, more preferably at least 32% HCl, more preferably at least 34% HCl, more preferably at least 36% HCl, or even more preferably a fuming HCl solution including between about 36.5% and 37.5% HCl. The amount of PCl₃ dissolved is preferably so that the concentration of PCl₃ in the solution is between about 0.5 M and about 1 M. Dissolution of PCl₃ in an aqueous HCl solution is exothermic.

The concentration of 6,6-dimethylhept-1-en-4-yn-3-ol (V) in the 6,6-dimethylhept-1-en-4-yn-3-ol (V) solution is preferably between about 2 M and about 10 M. Preferably the solution comprises ethanol as a solvent.

After the reaction of the 6,6-dimethylhept-1-en-4-yn-3-ol (V) with the PCl₃/HCl solution is completed, the resulting 1-chloro-6,6-dimethyl-2-hepten-4-yne (IVb) is preferably extracted from the aqueous reaction medium using an organic extraction solution including at least one organic solvent. A preferred organic extraction solution comprises hexane. Hexane effectively extracts 1-chloro-6,6-dimethyl-2-hepten-4-yne (IVb) from the aqueous reaction medium while extracting little or no by-products. A 1-chloro-6,6-dimethyl-2-hepten-4-yne (IVb)/hexane solution is easily washed and dried using conventional techniques. Subsequently, the hexane is evaporated, leaving a 1-chloro-6,6-dimethyl-2-hepten-4-yne (IVb) residue that is sufficiently pure for most uses without further processing. Evaporation of hexane requires little energy and the evaporated hexane is easily reprocessed for further use.

The isolated 1-chloro-6,6-dimethyl-2-hepten-4-yne (IVb) can be stored or reacted with N-methyl-1-naphthylmethyl amine (III) to yield Terbinafine (I).

Thus, according to this process of the present invention reaction of purified 6,6-dimethylhept-1-en-4-yn-3-ol (V) with a chlorination reagent is efficient, cheap and clean, allowing both storage and in situ use of the 1-chloro-6,6-dimethyl-2-hepten-4-yne (IVb) obtained without complex purification steps. Purification of 6,6-dimethylhept-1-en-4-yn-3-ol (V) produced using the teachings of U.S. Pat. No. 6,570,044, which are also taught herein, is relatively simple, so little is gained by removing this step.

When the process of the present invention is performed using a mixture of the two 1-chloro-6,6-dimethyl-2-hepten-4-yne (IVb) isomers, the yield of Terbinafine (I) relative to acrolein is at least similar and even higher than processes based on isolating the trans 1-chloro-6,6-dimethyl-2-hepten-4-yne (IVb). Thus, prior art processes including separation of the two 1-chloro-6,6-dimethyl-2-hepten-4-yne (IVb) isomers offer no advantages.

As is clear to one skilled in the art, the improved process of the present invention is highly efficient and is more applicable to the industrial scale preparation of Terbinafine (I) than the teachings of the prior art.

As concerns the reaction of 6,6-dimethylhept-1-en-4-yn-3-ol (V) to provide 1-chloro-6,6-dimethyl-2-hepten-4-yne (IVb) the following features, amongst others, are advantageous:

The process of the present invention teaches the use of a pre-prepared solution of a chlorination reagent, which is more efficient in an industrial setting;

The process is performed in a cheap and easy to handle ethanolic aqueous reaction medium, which is more environmentally friendly compared to other used processes.

The process is performed under regular conditions, such that an inert atmosphere is not required;

The process is performed at temperatures higher than 0° C.;

The process involves energy savings due to the reduced reaction time (only two hours); and

The yields relative to acrolein are higher as compared with other processes described in the art.

As concerns the reaction of 1-chloro-6,6-dimethyl-2-hepten-4-yne (IVb) with N-methyl-1-naphtylmethylamine (III) to provide Terbinafine (I) the following features, amongst others, are advantageous:

The process is performed in a tap water aqueous reaction medium which is cheap, easy to handle and environmentally friendly, and further allows extraction into cheap and easy to recycle organic solvent such as toluene that subsequently affords high yield isolation of Terbinafine (I), that conforms to pharmaceutical quality;

The process is performed under regular conditions, such that an inert atmosphere is not required;

The process does not require a catalyst;

The process uses cheap and safe inorganic basic salts such as potassium carbonate or sodium carbonate rather than requiring the use of hazardous organic amines as bases;

The process is performed, at all of its stages, at temperatures higher than 0° C. and further does not involve procedures in which combination of reagents is performed at reduced temperatures; and

The process is performed at relatively low temperatures, not exceeding 80° C.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description illustrate the invention in a non-limiting fashion.

Generally, the nomenclature used herein and the laboratory procedures utilized in the present invention include chemical and analytical techniques with which one skilled in the art is familiar. Unless otherwise defined, technical and scientific terms used herein have the same meaning as commonly understood by one of ordinary skill in the art to which this invention belongs. Although processes and materials similar or equivalent to those described herein can be used in the practice or testing of the present invention, suitable processes and materials are described below.

### Example 1

### Preparation of 1-chloro-6,6-dimethyl-2-heptene-4-yne

6,6-dimethylhept-1-en-4-yn-3-ol (V) was prepared according to the procedure described U.S. Pat. No. 6,570,044.

72.5 ml of a 36% aqueous HCl solution and 8 grams (0.058 mole) phosphorous trichloride were combined with gentle mechanical stirring in a three-necked 250 ml reactor. The temperature of the mixture rose to 40° C. Mixing was continued at 40° C. until a clear solution was obtained. The reaction mixture was cooled to 10° C. and a solution of 20 grams (0.145 mole) 6,6-dimethylhept-1-en-4-yn-3-ol (V) in 23 ml of ethanol was added. With continuous mixing, the mixture was gradually warmed up to and maintained at room temperature for a period of two hours.

30 ml hexane and 30 ml ice-cold water were added and the two phases separated. The organic phase was washed with a 10% sodium bicarbonate solution (3×30 ml) followed by multiple water washes to neutrality. The organic phase was dried with magnesium sulfate and the solvent evaporated under reduced pressure to obtain 22.1 gram (0.141 mole, 97.5% yield relative to V) of crude l-chloro-6,6-dimethyl-2-heptene-4-yne (IVb) having a 3.5:1 trans/cis ratio, as determined by GC as follows:

50 mg of product were dissolved in 3.0 ml ethanol and 15 ml diethylamine in a 25 ml volumetric flask. The flask was heated in an oven to 50° C. for one hour. Additional ethanol was added to the flask to complete the 25 ml volume.

A sample of the thus prepared solution was injected into a gas chromatograph using a FID detector and a split-mode injector on a Teknokroma column (Cat. Nr. TR-250233, TRB-225, 30 m, ID=0.32 mm, film thickness 0.25 microns). The conditions used for chromatographic separation were: 60° C. initial column temperature, 2 minutes hold time, final column temperature 220° C., heating rate 10° C. min⁻¹, hold time 15 minutes, injector temperature 250° C., detector temperature 250° C., carrier gas helium, column flow 1.0 ml min⁻¹, injection volume 0.5 μl and a split ratio of 1:60.

Under these conditions, the trans isomer was observed to have a retention time of 10.35 minutes and the cis isomer was observed to have a retention time of 8.65 minutes. The ratio of the areas of the detected peaks was taken to represent the relative amounts in the sample of the respective compounds.

### Example 2

### Preparation of Terbinafine HCl Using Aqueous HCl

124.8 grams (0.601 mole) N-methyl-1-naphthylmethyl amine (III) HCl followed by 120 grams (1.1 mole) sodium carbonate were added to 720 ml tap water in a three-necked reactor with stirring at 300-350 rpm. The reaction mixture was heated to 77-83° C. 93.6 grams (0.598 mole) 1-chloro-6,6-dimethyl-2-heptene-4-yne (IVb), obtained as described above were added over a four-hour period. After 4 additional hours at 80° C., stirring was ceased, leading to an immediate appearance of two phases. The lower aqueous phase was removed from the reactor and washed with toluene (2×100 ml). The two toluene washes and an additional 720 ml toluene were added to the reactor. The toluene solution was allowed to cool to room temperature.

66 ml of a 32% aqueous HCl solution were added to the toluene solution so as to acidify the solution to a pH of about 0.5-1.5 as measured using a Gel Pressure Electrode, produced by Mettler-Toledo International. After 20 minutes, 200 ml water were added, and the resulting suspension was stirred at 20-30° C. for 15 minutes and then filtered using Whatman No. 1 filter paper. The cake was washed with toluene (3×120 ml) and with water (1×150 ml). The damp cake was dried at 50° C. for 5 hours, to give 174 gram of a mixture containing the hydrochloride salts of Terbinafine (I) and the Terbinafine cis isomer (II) (0.555 mole, 93.8% yield relative to IVb).

### Example 3

### Preparation of Terbinafine HCl Using Gaseous HCl

124.8 grams (0.601 mole) N-methyl-1-naphthylmethyl amine (III) HCl followed by 120 grams (1.1 mole) sodium carbonate were added to 720 ml tap water in a three-necked reactor with stirring at 300-350 rpm. The reaction mixture was heated to 77-83° C. 93.6 grams (0.598 mole) 1-chloro-6,6-dimethyl-2-heptene-4-yne (IVb) (3.5:1 trans/cis ratio) were added over a four-hour period. After 4 additional hours at 80° C., stirring was ceased, leading to an immediate appearance of two phases. The lower aqueous phase was removed from the reactor and washed with toluene (2×100 ml). The two toluene washes and an additional 720 ml toluene were added to the reactor. The toluene solution was allowed to cool to room temperature.

Gaseous HCl was bubbled through the solution over a period of 20 minutes until the pH reached 1.5 as measured using a Gel Pressure Electrode, produced by Mettler-Toledo International. The reaction was exothermic, the temperature of the solution rising from 30° C. to 50° C. in 30 minutes. The reaction was allowed to cool to room temperature. After 20 minutes, 200 ml water were added, and the resulting suspension was stirred at 20-30° C. for 15 minutes and then filtered using Whatman No. 1 filter paper. The cake was washed with toluene (3×120 ml) and water (1×150 ml). The damp cake was dried at 50° C. for 5 hours, to give 182 grams of a mixture containing the hydrochloride salts of Terbinafine (I) and the Terbinafine cis isomer (II) (0.580 mole, 97.1% yield relative to IVb).

### Example 4

### Preparation of Purified Terbinafine HCl

100 grams of the product from Example 2, containing the hydrochloride salt of Terbinafine (I) and the cis isomer (II) was dissolved in 500 ml isopropanol at reflux temperature. The solution was cooled to 25° C. and the mixture was stirred for 4 hours. The resulting suspension was filtered using Whatman No. 1 filter paper and the cake washed with 60 ml isopropyl alcohol. After 4 hours drying at 50° C., 80 grams of Terbinafine hydrochloride were obtained, having a purity greater than 99.5%, as determined by HPLC analysis as follows:

A mobile phase comprising 200 ml THF, 400 ml acetonitrile and 400 ml of an aqueous buffer solution (6 g sodium dihydrogen phosphate dihydrate in 1 liter of water and adjusted to pH 6.5 using 1.0 N NaOH) was prepared. 30 mg of product was dissolved in 100.0 ml of the mobile phase.

A 10 μl sample of the thus prepared product-containing solution into an HPLC using a UV detector at 220 nm on a 250×4.6 mm Luna C8(2) column (5 micron, available from Phenomenex Cat. Nr. 00G-4249-EO). The conditions used for chromatographic separation were: 45° C. oven temperature, flow rate of 1.0 ml min⁻¹ using the mobile phase described above.

Under these conditions, Terbinafine (I) was observed to have a retention time of 27.6 minutes and the respective cis isomer (II) was observed to have a relative retention time of 0.89. Other potential impurities include 4-methyl Terbinafine (relative retention time) 1.21 and β-Terbinafine (0.82). The ratio of the areas of the detected peaks was taken to represent the relative amounts in the sample of the respective compounds.

### Example 5

### Large-Scale Preparation of Terbinafine HCl

100 kilograms (482 mole) N-methyl-1-naphthylmethyl amine (III) HCl followed by 95 kilograms (800 mole) sodium carbonate were added to 550 liter tap water in a 1000 liter reactor with stirring. The reaction mixture was heated to 77-83° C. 75 kilogram (479 mole) 1-chloro-6,6-dimethyl-2-heptene-4-yne (IVb) (4:1 trans/cis ratio) were added over a four hour period. After 2 additional hours at 77-83° C., stirring was ceased, leading to the appearance of two phases. The lower aqueous phase was removed, 570 liters toluene were added to the reactor, mixed for 15 minutes, and removed. The aqueous phase was returned to the reactor and mixed with 80 liters toluene for 15 minutes. After an additional 15 minutes, the aqueous phase was removed and the previously removed product-containing toluene solution returned to the reactor. The toluene solution was allowed to cool to 20° C.-25° C.

60 liters of a 32% aqueous HCl solution were added to the toluene solution, so as to acidify the solution over a period of two hours while the temperature was maintained at 20° C.-25° C. Once the addition of HCl was completed, it was confirmed that the pH of the solution was less than 1.5, and mixing continued for an additional 15 minutes. The suspension was then filtered (using a filter pressure). The cake was washed with 100 liters toluene, then with 200 liters water and then with another 100 liters toluene. The damp cake was dried at 50° C. for 12 hours under a nitrogen stream to obtain 140 kilogram of a mixture containing the hydrochloride salts of Terbinafine (I) and the Terbinafine cis isomer (II) (446 mole, 93.1% yield relative to IVb).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

The present invention is not to be limited in scope by the specific embodiments described herein. Indeed, various modifications of the invention in addition to those described herein will become apparent to those skilled in the art from the foregoing description and the accompanying schemes. Such modifications are intended to fall within the scope of the appended claims.

Patents, patent applications, publications, procedures, and the like are cited throughout this application, the disclosures of which are incorporated herein by reference in their entireties. To the extent that a conflict may exist between the specification and a reference, the language of the disclosure made herein controls.

What is claimed is:

1. A process of preparing Terbinafine and/or a salt thereof, the process comprising:
providing 1-chloro-6,6-dimethyl-2-hepten-4-yne; and
reacting said 1-chloro-6,6-dimethyl-2-hepten-4-yne with N-methyl-1-naphtylmethylamine, in an aqueous reaction medium, thereby obtaining said Terbinafine.

2. The process of claim 1, further comprising, subsequent to said reacting:
contacting said Terbinafine with HCl, to thereby obtain a Terbinafine HCl salt as a precipitate.

3. The process of claim 2, further comprising:
re-crystallizing said Terbinafine HCl salt, to thereby obtain Terbinafine HCl salt having a pharmaceutical quality.

4. The process of claim 3, further comprising:
converting said Terbinafine HCl salt having a pharmaceutical quality into Terbinafine, thereby obtaining said pure Terbinafine having a pharmaceutical quality.

5. The process of claim 1, wherein said aqueous reaction medium comprises at least 25% by weight water.

6. The process of claim 1, wherein said reacting comprises:
providing an aqueous solution containing said N-methyl-1-naphtylmethylamine; and
reacting said 1-chloro-6,6-dimethyl-2-hepten-4-yne with said aqueous solution containing said N-methyl-1-naphtylmethylamine.

7. The process of claim 1, wherein said aqueous reaction medium comprises a base.

8. The process of claim 7, wherein said base is an inorganic base selected from the group consisting of sodium carbonate, potassium carbonate, sodium bicarbonate and potassium bicarbonate.

9. The process of claim 7, wherein said base is sodium carbonate.

10. The process of claim 7, wherein the concentration of said base ranges between about 50 and about 400 gram per liter of said aqueous reaction medium.

11. The process of claim 1, wherein the molar ratio between said 1-chloro-6,6-dimethyl-2-hepten-4-yne and said N-methyl-1-naphtylmethylamine ranges between about 1:0.9 and 1:1.1.

12. The process of claim 6, wherein a concentration of said N-methyl-1-naphthylmethyl amine in said aqueous solution ranges between about 0.1 M and about 20 M.

13. The process of claim 6, wherein said reacting is effected by adding said 1-chloro-6,6-dimethyl-2-hepten-4-yne to said aqueous solution.

14. The process of claim 6, further comprising:
prior to said reacting, heating said aqueous solution to a temperature of at least 40° C.

15. The process of claim 1, further comprising, subsequent to said reacting:
providing an organic solution containing said Terbinafine.

16. The process of claim 15, wherein providing said organic solution containing said Terbinafine comprises:
separating a first amount of said Terbinafine from said aqueous reaction medium.

17. The process of claim 16, wherein providing said organic solution containing said Terbinafine further comprises, subsequent to said separating:
contacting said aqueous reaction medium with an organic extraction solution, to thereby extract said Terbinafine into said organic extraction solution.

18. The process of claim 17, wherein said organic extraction solution comprises at least one organic solvent selected from the group consisting of an ether, a linear alkane, a cycloalkane, a branched alkane, an aromatic solvent, an ester, a ketone, a halogenated hydrocarbon, a nitrile and any mixture thereof.

19. The process of claim 18, wherein said at least one organic solvent comprises toluene.

20. The process of claim 17, wherein providing said organic solution containing said Terbinafine further comprises:
combining said Terbinafine and said organic extraction solution, to thereby obtain said organic solution containing said Terbinafine.

21. The process of claim 2, further comprising, prior to said contacting and subsequent to said reacting:
providing an organic solution containing said Terbinafine.

22. The process of claim 21, wherein contacting said Terbinafine with HCl comprises contacting said organic solution containing said Terbinafine with an aqueous HCl solution.

23. The process of claim 22, wherein said aqueous HCl solution further comprises ethanol.

24. The process of claim 20, wherein contacting said Terbinafine with HCl comprises contacting said organic solution containing said Terbinafine with gaseous HCl.

25. The process of claim 3, wherein said re-crystallizing is performed in an organic solvent.

26. The process of claim 25, wherein said organic solvent is selected from the group consisting of methanol, ethanol, n-propanol, isopropanol, n-butanol, sec-butanol, ethyl acetate, isopropyl acetate, n-butyl acetate, isobutyl acetate, acetonitrile and any mixture thereof.

27. The process of claim 25, wherein said organic solvent comprises isopropanol.

28. The process of claim 1 wherein providing said 1-chloro-6,6-dimethyl-2-hepten-4-yne comprises:
providing 6,6-dimethylhept-1-en-4-yn-3-ol; and
reacting said 6,6-dimethylhept-1-en-4-yn-3-ol with a chlorinating agent, in an aqueous reaction medium, to thereby obtain said 1-chloro-6,6-dimethyl-2-hepten-4-yne.

29. The process of claim 28, wherein said providing said 6,6-dimethylhept-1-en-4-yn-3-ol comprises:
providing t-butylacetylide; and
reacting said t-butylacetylide with acrolein.

30. The process of claim 29, wherein providing said t-butylacetylide comprises reacting t-butylacetylene and an organomagnesium compound of the Grignard type.

31. The process of claim 30, wherein said organomagnesium compound is ethylmagnesium bromide.

32. The process of claim 29, wherein said reacting is performed at a temperature of between about 0° C. and about 5° C.

33. The process of claim 28, wherein said chlorinating agent comprises a mixture of PCl₃ and HCl.

34. The process of claim 28, wherein said reacting is performed at a temperature greater than about 0° C.

35. The process of claim 28, further comprising, prior to said reacting:
providing a solution containing said chlorinating agent.

36. The process of claim 35, wherein said aqueous reaction medium comprises at least one water-miscible organic solvent.

37. The process of claim 36, wherein said water-miscible organic solvent comprises ethanol.

38. The process of claim 35, wherein said solution comprises at least 25% by weight water.

39. The process of claim 28, further comprising:
contacting said aqueous reaction medium with an organic extraction solution, to thereby provide an organic solution containing said 1-chloro-6,6-dimethyl-2-hepten-4-yne.

40. A process of preparing l-chloro-6,6-dimethyl-2-hepten-4-yne, the process comprising:
providing 6,6-dimethylhept-1-en-4-yn-3-ol; and
reacting said 6,6-dimethylhept-1-en-4-yn-3-ol with a chlorinating agent, in an aqueous reaction medium, to thereby obtain the 1-chloro-6,6-dimethyl-2-hepten-4-yne.

41. The process of claim 40, wherein said providing said 6,6-dimethylhept-1-en-4-yn-3-ol comprises:
providing t-butylacetylide; and
reacting said t-butylacetylide with acrolein.

42. The process of claim 41, wherein providing said t-butylacetylide comprises reacting t-butylacetylene and an organomagnesium compound of the Grignard type.

43. The process of claim 42, wherein said organomagnesium compound is ethylmagnesium bromide.

44. The process of claim 41, wherein said reacting is performed at a temperature of between about 0° C. and about 5° C.

45. The process of claim 40, further comprising, prior to said reacting, providing a solution containing said 6,6-dimethylhept-1-en-4-yn-3-ol and a water miscible organic solvent.

46. The process of claim 45, wherein said water miscible organic solvent is selected from the group consisting of methanol, ethanol, n-propanol, isopropanol, n-butanol, sec-butanol and any mixture thereof.

47. The process of claim 45, wherein a concentration of said 6,6-dimethylhept-1-en-4-yn-3-ol in said solution ranges between about 0.1 and about 20 M.

48. The process of claim 40, wherein said chlorinating agent comprises a mixture of PCl₃ and HCl.

49. The process of claim 48, further comprising, prior to said reacting:
providing a solution including HCl and PCl₃.

50. The process of claim 49, wherein said solution comprises water.

51. The process of claim 49, wherein said solution comprises ethanol.

52. The process of claim 49, wherein a concentration of said PCl₃ in said solution is between about 0.1 M and about 3 M.

53. The process of claim 49, wherein a concentration of said HCl in said solution is greater than about 20%.

54. The process of claim 40, wherein said reacting is performed at a temperature greater than about 0° C.

55. The process of claim 40, further comprising, prior to said reacting:
providing a solution containing said chlorinating agent.

56. The process of claim 55, wherein said aqueous reaction medium comprises at least one water-miscible organic solvent.

57. The process of claim 56, wherein said water-miscible organic solvent is selected from the group consisting of methanol, ethanol, n-propanol, isopropanol, n-butanol, sec-butanol and any mixture thereof.

58. The process of claim 56, wherein said water-miscible organic solvent comprises ethanol.

59. The process of claim 55, wherein said solution comprises at least 25% by weight water.

60. The process of claim 40, further comprising:
contacting said aqueous reaction medium with an organic extraction solution, to thereby provide an organic solution containing said 1-chloro-6,6-dimethyl-2-hepten-4-yne.

61. The process of claim 60, wherein said organic extraction solution comprises at least one organic solvent selected from the group consisting of ethers, linear alkanes, cycloalkanes, branched alkanes, aromatic solvents, esters, ketones, halogenated hydrocarbons, nitrites and any mixture thereof.

62. The process of claim 61, wherein said organic extraction solution comprises hexane.
